(19) United States
(12) Patent Application Publication
(10) Pub. No.: US 2005/0165011 A1
(43) Pub. Date: Jul. 28, 2005

(54) Benzoxazine and benzoxazinone substituted triazoles

(76) Inventors: Francoise Jeanne Gellibert, Les Ulis (FR); Andrew H. Payne, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/514,091

(22) PCT Filed: May 13, 2003
(86) PCT No.: PCT/GB03/02049

(30) Foreign Application Priority Data
May 15, 2002 (GB) 0211132.6
Jul. 31, 2002 (GB) 0217754.1

(51) Int. Cl. A61K 31/538; C07D 413/14

(52) U.S. Cl. 514/230.5; 544/105

(57) ABSTRACT

This invention relates to benzoxazine and benzoxazinone substituted triazoles which are inhibitors of the transforming growth factor, (“TGF”)-β signalling pathway, in particular, the phosphorylation of smad2 or smad3 by the TGF-β, type I or activin-like kinase (“ALK”)-5 receptor, methods for their preparation and their use in medicine, specifically in the treatment and prevention of a disease state mediated by this pathway.

### COMPOUNDS

This invention relates to benzoxazine and benzoxazinone substituted triazoles which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signalling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasminogen activator inhibitor-i, components of sclerotic deposits, through the action of smad3 phosphorylation by the TGF-β1 receptor ALK5. Zhang Y., et al, Nature, 1998; 394(6696), 909-13; Usui T., etal, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest, 1993; 68(2), 154-63, diabetic nephropathy Yamamoto, T., et al, 1993, PNAS 90, 1814-1818, allograft rejection, HIV nephropathy and angiotensin-induced nephropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest, 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest, 1995; 96(6), 2667-75. TGF-β1 was immunolocalized to non-foamy macrophages in atherosclerotic lesions where active matrix synthesis occurs,. suggesting that non-foamy macrophages may participate in modulating matrix gene expression in atherosclerotic remodeling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralising antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signalling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

Surprisingly, it has now been discovered that a class of benzoxazine and benzoxazinone substituted triazole compounds function as potent and selective non-peptide inhibitors of ALK5 kinase and therefore, have utility in the treatment and prevention of various disease states mediated by ALK5 kinase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

According to a first aspect, the invention provides a compound of formula (I) or a pharmaceutically acceptable derivative thereof: 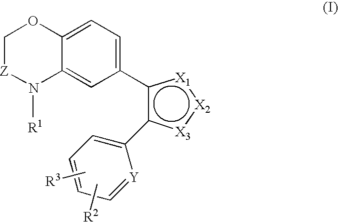
wherein Z is CH₂ or C═O;
Y is N or CH;
R¹ is selected from H, C₁₋₆alkyl, C₂₋₆alkenyl, —(CH₂)ₚ—NR⁴R⁵, —(CH₂)ₚ—OR⁴, —(CH₂)ₚ—CN, —(CH₂)P—CONR⁴R⁵, —(CH₂)ₚ—NHCOR⁴₁ —(CH₂)ₚ—NHSO₂R⁴ and —(CH₂)ₚ-het, wherein the het group is optionally substituted by C₁₋₆alkyl; or when Z is CH₂, R¹ may additionally be selected from —CO—C₁₋₆alkyl, —CO—(CH₂)_{q}OR⁴, —CO—(CH₂)_{q}—NR⁴R⁵ and —CO—(CH₂)_{q}-het wherein the het group is optionally substituted by C₁₋₆alkyl;
R² is selected from H, C₁₋₆alkyl, halo, CN or perfluoroC₁₋₆alkyl;
R³ is selected from H or halo;
R⁴ and R⁵ are independently selected from H or C₁₋₆alkyl; or R⁴ and R⁵ together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₄ alkyl and C1-4 alkoxy;
two of X₁, X₂ and X₃ are N and the other is NR⁶ wherein R⁶ is hydrogen, C₁₋₆alkyl, C₃₋₇cycloalkyl, —(CH₂)ₚ—CN, —(CH₂)ₚ—CO₂H, —(CH₂)ₚ—CONR⁷R⁸, —(CH₂)PCOR⁷, —(CH₂)_{q}(OR⁹)₂, —(CH₂)ₚOR⁷, —(CH₂)_{q}—CH═CH—CN, —(CH₂)_{q}—CH═CH—CO₂H, —(CH₂)_{q}CH═CH—CONR⁷R⁸, —(CH₂)ₚNHCOR¹⁰ or —(CH₂)ₚNR¹¹R¹²;
R⁷ and R³ are independently H, C₁₋₆alkyl, aryl or het;
R⁹ is C₁₋₆alkyl;
R¹⁰ is C₁₋₇alkyl, aryl, heteroaryl, arylC₁₋₆alkyl or heteroarylC₁₋₆alkyl;
R¹¹ and R¹² are independently selected from hydrogen, C₁₋₆alkyl, aryl and arylC₁₋₆alkyl, or R¹¹ and R¹² together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, OCF₃, Cl4 alkyl and C₁₋₄ alkoxy;
p is 2-4; and
q is 1-4.

In the triazole ring of the compounds of formula (I) it will be apparent that there will be a double bond between the two unsubstituted nitrogens.

Preferably Y is N.

Preferably, R¹ is H, C₁₋₆alkyl, C₂₋₆alkenyl, —(CH₂)₂-Het, —(CH₂)₂—OR⁴, —(CH₂)₂—NR⁴R⁵ or —(CH₂)₂—CN. More preferably R¹ is H, C₁₋₆alkyl or C₂6alkenyl.

Preferably, R² is H, C₁alkyl or halo. More preferably, R² is H, methyl, chloro or fluoro. Preferably, when Y is N, R² is methyl positioned ortho to Y.

Preferably, R³ is H or halo. More preferably, R³ is H or fluoro. Most preferably, when Y is N and R² is methyl positioned ortho to Y, R³ is H.

Preferably, R⁴ and R⁵ are independently H or methyl, or R⁴ and R⁵ together with the atom to which they are attached form a 3, 4, 5, 6 or 7 membered saturated or unsaturated ring which may contain one or more heteroatoms selected from N, S or O, and wherein the ring may be further substituted by one or more substitutents selected from halo (such as fluoro, chloro, bromo), —CN, —CF₃, —OH, —OCF₃, C₁₋₄ alkyl and C₁₋₄ alkoxy. Suitably, R⁴ and R⁵ together with the atom to which they are attached form a morpholine, piperidine, pyrrolidine, piperazine or N-methyl piperazine ring.

Preferably, R⁶ is H.

It will be appreciated that the present invention is intended to include compounds having any combination of the preferred groups listed hereinbefore.

Compounds of formula (I) which are of special interest as agents useful in the treatment or prophylaxis of disorders characterised by the overexpression of TGF-β are:
6-[5-(6-methyl-pyridin-2-yl)-1H-[1,2,3]triazol-4-yl]4H-benzo[1,4]oxazin-3-one (Example 1);
6-(5-pyridin-2-yl-1H-[1,2,3]triazol4-yl)-4H-benzo[1,4]oxazin-3-one (Example 2);
6-[5-(3-chloro-phenyl)-1H-[1,2,3]triazol-4-yl]-4H-benzo[1,4]oxazin-3-one (Example 3);
4-methyl-6-[5-(6-methyl-pyridin-2-yl)-1H-[1,2,3]triazol-4-yl]4H-benzo[1,4]oxazin-3-one (Example 4);
4-ethyl-6-[5-(6-methyl-pyridin-2-yl)-1H-[1,2,3]triazol-4-yl]-4H-benzo[1,4]oxazin-3-one (Example 5);
4-propyl-6-[5-(6-methyl-pyridin-2-yl)-1H-[1,2,3]triazol-4-yl]4H-benzo[1,4]oxazin-3-one (Example 6);
4-(propen-2-yl)-6-[5-(6-methyl-pyridin-2-yl)-1H-[1,2,3]triazol-4-yl]-4H-benzo[1,4]oxazin-3-one (Example 7);
4-(2-methoxy-ethyl)-6-[5-(6-Methyl-pyridin-2-yl)-1H-[1,2,3]triazol-4-yl]-4H-benzo[1,4]oxazin-3-one (Example 8);
3-{6-[5-(-6-methyl-pyridin-3-yl)-1H-[1,2,3]triazol-4-yl]-3-oxo-2,3-dihydro-benzo[1,4]oxazin4-yl}-propionitrile (Example 9);
6-[5-(6-methyl-pyridin-2-yl)-1H-[1,2,3]triazol4-yl]4-(2-morpholin-4-yl-ethyl)4H-benzo[1,4]oxazin-3-one (Example 10);
4-(2-dimethylamino-ethyl)-6-[5-(6-Methyl-pyridin-2-yl)-1H-[1,2,3]triazol-4-yl]-4-(2-morpholin-4-yl-ethyl)4H-benzo[1,4]oxazin-3-one (Example 1 1);
6-[5-(6-methyl-pyridin-2-yl)-1H-[1,2,3]-triazol-4-yl]-3,4-dihydro-2H-benzo[1,4]-oxazine (Example 15);
4-methyl-6-[5-(6-methyl-pyridin-2-yl)-1H-[1,2,3]-triazol-4-yl]-3,4-dihydro-2H-benzo[1,4]-oxazine (Example 13);
4-acetyl-6-[5-(6-methyl-pyridin-2-yl)-1H-[1,2,3]-triazol-4-yl]-3,4-dihydro-2H-benzo[1,4]-oxazine (Example 14);
4-ethyl-6-[5-(6-methyl-pyridin-2-yl)-1H-[1,2,3]-triazol-4-yl]-3,4-dihydro-2H-benzo[1,4]-oxazine (Example 12);
4-(propen-2-yl)-6-[5-(6-methyl-pyridin-2-yl)-1H-[1,2,3]-triazol-4-yl]-3,4-dihydro-2H-benzo[1,4]-oxazine (Example 16); and
4-[(4-methyl-1-piperazinyl)acetyl]-6-(6-methyl-pyridin-2-yl-1H-[1,2,3]-triazol4-yl)-3,4 dihydro-2H-benzo[1,4]-oxazine (Example 17) or pharmaceubcally acceptable derivatives thereof.

According to a further aspect, the invention provides a compound of formula (I) or a pharmaceutically acceptable derivative thereof: 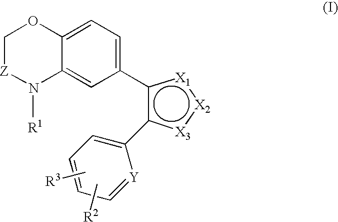
wherein Z is CH₂ or C═O;
Y is N or CH;
R¹ is selected from H, C₁₋₆alkyl, C₁₋₆alkenyl, —(CH₂)ₚ—NR⁴R⁵, —(CH₂)P—OR⁴, —(CH₂)ₚ—CN, —(CH₂)P—CONR⁴R⁵, —(CH₂)P—NHCOR⁴, —(CH₂)ₚ—NHSO₂R⁴, —(CH₂)ₚ—Het; or when Z is CH₂, R¹ may additionally be selected from —CO—C₁₋₆alkyl, —CO—(CH2)_{q}-15 OR⁴, —CO—(CH₂)_{q}—NR⁴R⁵, —CO—(CH₂)_{q}-Het;
R² is selected from H, C₁₋₆alkyl, halo, CN or perfluoroC₁₋₆alkyl;
R³ is selected from H or halo;
R⁴ and R⁵ are independently selected from H or C₁₋₆alkyl; or R⁴R⁵ together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₄ alkyl and C₁₋₄ alkoxy;
two of X₁, X₂ and X₃ are N and the other is NR⁶ wherein R⁶ is hydrogen, C₁₋₄alkyl, C₃₋₇Cycloalkyl, —(CH₂)ₚ—CN, —(CH₂)ₚ—CO₂H, —(CH₂)ₚ—CONHR⁷R⁸, —(CH₂)ₚCOR⁷, —(CH₂)_{q}(OR⁹)2, —(CH₂)ₚOR⁷, —(CH₂)_{q}—CH═CH—CN, —(CH₂)_{q}CH═CH—CO₂H, —(CH₂)_{q}—CH═CH—CONHR⁷R⁸, —(CH₂)ₚNHCOR¹⁰ or —(CH₂)ₚNR¹¹R¹²;
R⁷ and R⁸ are independently H, C₁₋₆alkyl, aryl or het;
R⁹ is C₁₋₆alkyl;
R¹⁰ is C₁₋₇alkyl, or optionally substituted aryl, heteroaryl, arylC₁₋₆alkyl or heteroarylC₁₋₆alkyl;
R¹¹ and R¹² are independently selected from hydrogen, C₁₋₆alkyl, aryl and arylC₁₋₆alkyl, or R¹¹R¹² together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, OCF₃, C₁₋₄ alkyl and C₁₋₄ alkoxy;
p is 2-4; and
q is 1-4.

Certain of the compounds of formula (I) may exist in the form of optical isomers, e.g. diastereolsomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

The terms “C₁₋₆alkyl” and “C₁₋₇alkyl” as used herein, whether on their own or as part of a group, refer to a straight or branched chain saturated aliphatic hydrocarbon radical of 1 to 6 carbon atoms respectively, unless the chain length is limited thereto, including, but not limited to methyl, ethyl, n-propyl, isopropyl, n-butyl, sec-butyl, isobutyl, tert-butyl, pentyl and hexyl.

The term “alkenyl” as a group or part of a group refers to a straight or branched chain mono- or poly-unsaturated aliphatic hydrocarbon radical containing the specified number(s) of carbon atoms. References to “alkenyl” groups include groups which may be in the E- or Z-form or mixtures thereof.

The term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, iso-butoxy, sec-butoxy and tett-butoxy.

The term “aryl” as a group or part of a group refers to a carbocyclic aromatic radical containing the specified number(s) of carbon atoms, preferably from 5 to 14 carbon atoms, and more preferably from 5 to 10 carbon atoms, which may include bi- and tricyclic systems, optionally substituted with one or more substituents, which may be the same or different, selected from halo (such as fluoro, chloro, bromo), —CN, —CF₃, —OH, —OCF₃, C₁₋₄ alkyl and C₁₋₄ alkoxy. Such aryl groups include cyclopentadieny, phenyl or naphthyl.

The term “cycloalkyl” as a group or part of a group refers to a saturated carbocyclic radical containing the specified number of carbon atom(s), preferably from 3 to 14 carbon atoms, more preferably 3 to 10 carbon atoms, optionally substituted with one or more substituents, which may be the same or different, selected from halo (such as fluoro, chloro, bromo), —CN, —CF₃, —OH, —OCF₃, C₁₋₄ alkyl and C₁₋₄ alkoxy. Such groups in particular include cyclopropyl, cyclobutyl, cyclopentyl and cyclohexyl.

The term “het” or “heteroaryl” as a group or part of a group refers to a stable heterocyclic aromatic 6 to 14 membered monocyclic ring containing one or more hetero atoms independently selected from nitrogen, oxygen and sulfur, optionally substituted with one or more substituents, which may be the same or different, selected from halo (such as fluoro, chloro, bromo), —CN, —CF₃, —OH, —OCF₃, C₁₋₄ alkyl and C₁₋₄ alkoxy. Suitably the 6 to 14-membered heterocyclic moiety is selected from furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, quinazoline, quinoline, isoquinoline and ketal.

The term “perfluoroalkyl” as used herein includes compounds such as trifluoromethyl.

The terms “halo” or “halogen” are used interchangeably herein to mean radicals derived from the elements chlorine, fluorine, iodine and bromine.

As used herein the term “pharmaceutically acceptable derivative” means any pharmaceutically acceptable salt, solvate, ester or amide, or salt or solvate of such ester or amide, of the compound of formula (I), or any other compound which upon administration to the recipient is capable of providing (directly or indirectly) the a compound of formula (I) or an active metabolite or residue thereof, eg, a prodrug. Preferred pharmaceutically acceptable derivatives according to the invention are any pharmaceutically acceptable salts, solvates or prodrugs.

Suitable pharmaceutically acceptable salts of the compounds of formula (I) include acid salts, for example sodium, potassium, calcium, magnesium and tetraalkylammonium and the like, or mono- or di- basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

The term “ALK5 inhibitor” is used herein to mean a compound, other than inhibitory smads, e.g. smad6 and smad7, which selectively inhibits the ALK5 receptor preferentially over p38 or type II receptors.

The term “ALKS mediated disease state” is used herein to mean any disease state which is mediated (or modulated) by ALK5, for example a disease which is modulated by the inhibition of the phosphorylation of smad, 2/3 in the TGF-1 i& signaling pathway.

The term “ulcers” is used herein to include, but not to be limited to, diabetic ulcers, chronic ulcers, gastric ulcers, and duodenal ulcers.

The compounds of formula (I) can be prepared by art-recognised procedures from known or commercially available starting materials. If the starting materials are unavailable from a commercial source, their synthesis is described herein, or they can be prepared by procedures known in the art.

Specifically, compounds of formula (I) may be prepared as illustrated in Schemes 1 to 4.

More specifically, when Z is C═O, compounds of formula (I) may be synthesised according to the procedure outlined in Scheme 1. 6-Bromo4H-benzo[1,4]oxazin-3-one (II) (Mazharuddin, N.; Thyagarajan, G.; Indian J. Chem.; 7; 1969; 658-661) is coupled with trimethylsilylacetylene using a palladium catalyst, such as Pd(PPh₃)₄, in the presence of copper(l) iodide. It will be appreciated by the skilled person that other catalysts may be used in place of Pd(PPh₃)₄. Examples of such catalysts include but are not limited to PdCl₂(PPh₃)₂. The trimethylsilyl group is then removed under basic conditions, for example, potassium carbonate and the unmasked terminal acetylene derivative (IV) is coupled to a substituted aryl bromide via palladium catalysis using the same conditions as for the preparation of (Ill). The resulting benzoxazinone derivative (V) may be alkylated with a suitable alkylating agent, L-R¹ where L is a leaving group, e.g. I or Br or Cl, and R¹ is as hereinbefore described, in the presence of a base such as potassium carbonate to form the N-alkylated derivative (VI). The disubstituted acetylene (V) or (VI) is treated with trimethylsilylazide to afford a triazole (VII) or (VIII) which may be alkylated with a suitable alkylating agent, L-R⁶ where L is a leaving group, e.g. I, and R⁶ is as hereinbefore described, in the presence of a base such as potassium carbonate. The resulting isomers can be separated by chromatographic methods.

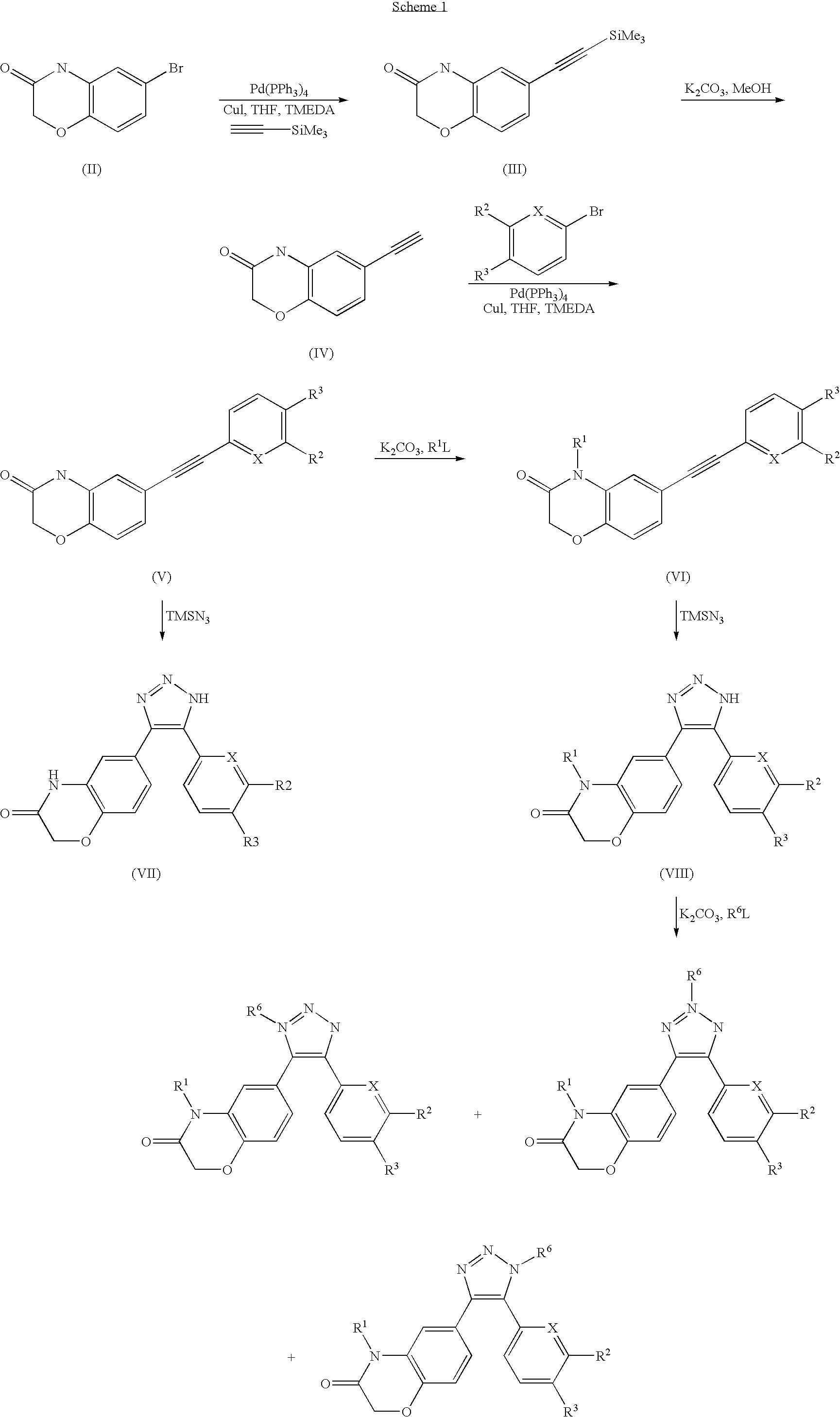

Compounds of formula (I) where Z is CH₂ and R⁶ is H may be prepared following the general method illustrated in Scheme 2. 4-Acetyl-3,4-dihydro-2H-10 benzo[b][1,4]oxazin-6-yl bromide (IX) (prepared as described in J. Med. Chem. 1997, 40, 1465-1474) is coupled with trimethylsilylacetylene using a palladium catalyst, for example Pd(PPh₃)₄, in the presence of copper(I) iodide to form the silyl derivative (X). The trimethylsilyl group is then removed under basic conditions, for example, potassium carbonate at room temperature and the unmasked terminal acetylene (XI) is coupled to a substituted aryl bromide via palladium catalysis under the same conditions as for the preparation of (X). The acetyl group is removed under basic conditions with potassium hydroxide. The resulting benzoxazine derivative (XIII) may be alkylated or acylated with a suitable agent, such as L-R¹ where L is a leaving group, e.g. I or Br or Cl, and R¹ is as hereinbefore described, in the presence of a base such as potassium carbonate or triethylamine to form the N-alkyl or N-acyl derivative (XIV). Alteratively, the benzoxazine derivative (XIII) may also be alkylated by reductive amination with R¹—CHO , in the presence of NaBH₃CN. The resulting disubstituted acetylene is treated with trimethylsilylazide to afford the triazole.

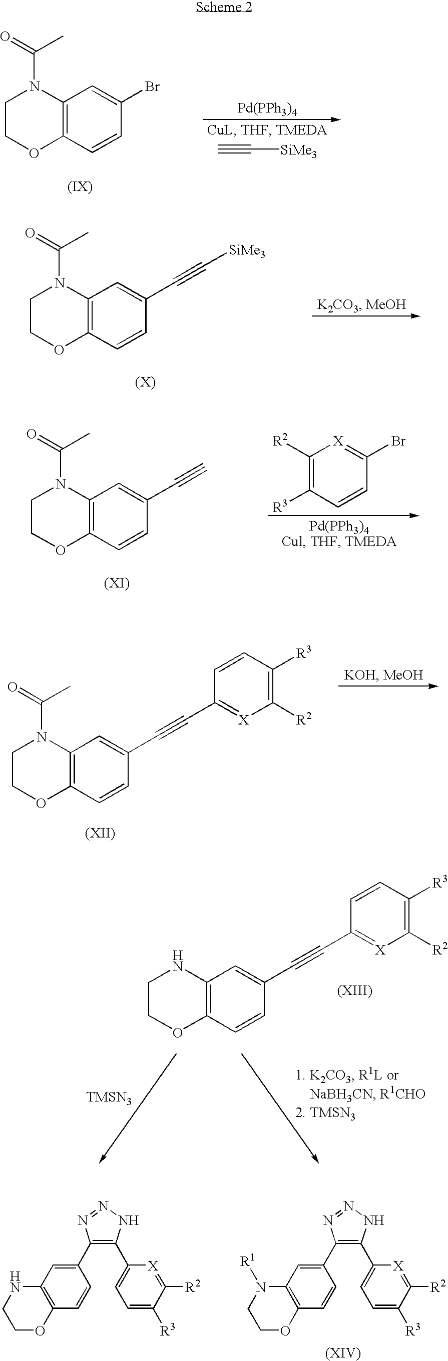

Compounds of formula (I) where R¹ is CO—CH2NR⁴R⁵ and R⁶ is H may be prepared as illustrated in Scheme 3.

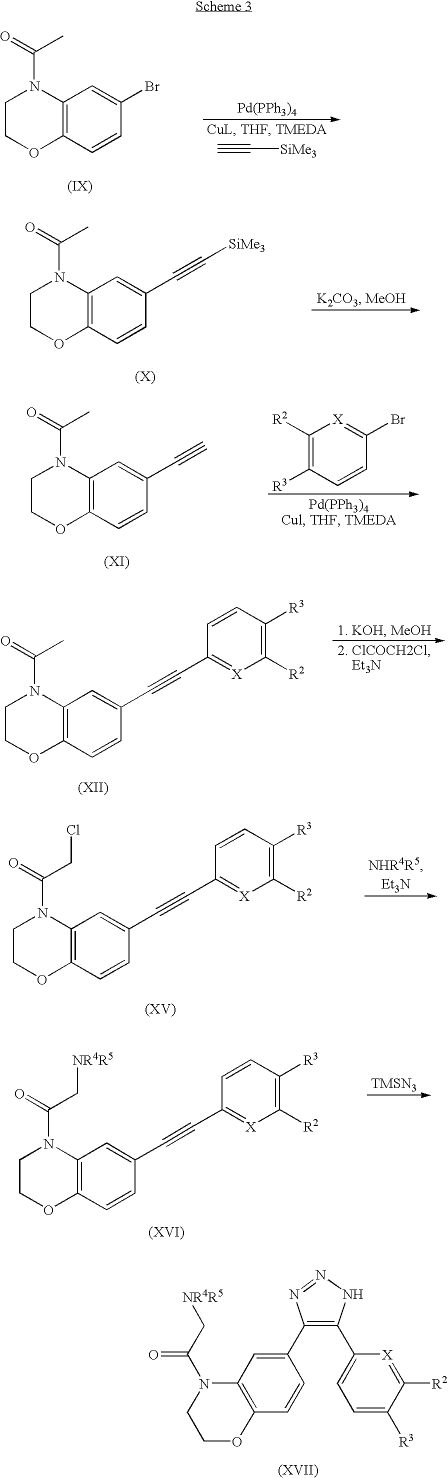

Benzoxazine compounds of formula (XIV) where R¹ is C₁₋₆alkyl, —(CH₂)₂-Het, or —(CH₂)2OR⁴, may be prepared from reduction of benzoxazinone compounds of formula (VI), as shown in reaction scheme 4.

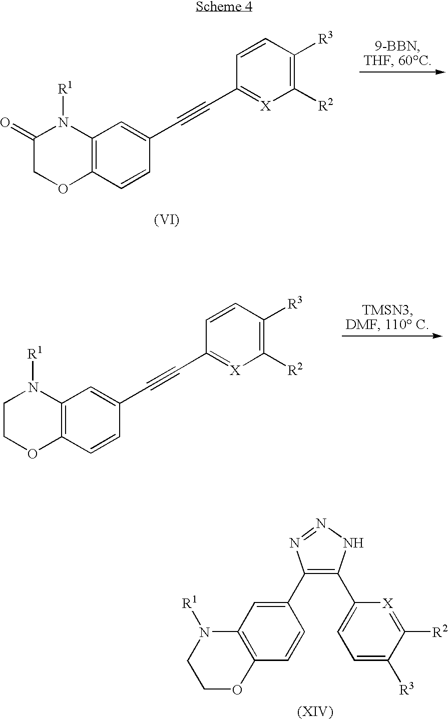

Further details for the preparation of compounds of formula (I) are found in the examples.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

The compounds of the present invention have been found to inhibit phosphorylation of the Smad-2 or Smad-3 proteins by inhibition of the TGF-β type I (ALK5) receptor.

Accordingly, the compounds of the invention have been tested in the assays described herein and have been found to be of potential therapeutic benefit in the treatment and prophylaxis of disorders characterised by the overexpression of TGF-β.

Thus, there is provided a compound of formula (I), or a pharmaceutically acceptable salt, solvate or derivative thereof, for use as a medicament in human or veterinary medicine, particularly in the treatment or prophylaxis of disorders characterised by the overexpression of TGF-β.

It will be appreciated that references herein to treatment extend to prophylaxis as well as the treatment of established conditions. It will further be appreciated that references herein to treatment or prophylaxis of disorders characterised by the overexpression of TGF-β, shall include the treatment or prophylaxis of TGF-β associated disease such as fibrosis, especially liver and kidney fibrosis, cancer development, abnormal bone function and inflammatory disorders and scarring.

Other pathological conditions which may be treated in accordance with the invention have been discussed in the introduction hereinbefore. The compounds of the present invention are particularly suited to the treatment of fibrosis and related conditions.

Compounds of the present invention may be administered in combination with other therapeutic agents, for example antiviral agents for liver diseases, or in combination with ACE inhibitors or Angiotensin II receptor antagonists for kidney diseases.

According to a further aspect of the present invention there is provided the use of a compound of formula (I) or a pharmaceutically acceptable salt thereof, in the manufacture of a medicament for the treatment of a disease mediated by the ALK5 receptor in mammals.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis, retroperitoneal fibrosis, mesenteric fibrosis, endometriosis, keloids and restenosis.

According to a further aspect of the present invention there is provided a method of inhibiting the TGF-β signaling pathway in mammals, for example, inhibiting the phosphorylation of smad2 or smad3 by the type I or activin-like kinase ALK5 receptor.

According to a further aspect of the present invention there is provided a method of inhibiting matrix formation in mammals by inhibiting the TGF-β signalling pathway, for example, inhibiting the phosphorylation of smad2 or smad3 by the type I or activin-like kinase ALK5 receptor.

The pharmaceutically effective compounds of formula (I) and pharmaceutically acceptable salts thereof, may be administered in conventional dosage forms prepared by combining a compound of formula (I) with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg perday.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

‘No toxicological effects are expected when a compound of formula (I) is administered in the above mentioned dosage range.’

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects where, unless otherwise stated, the compound of formula (I) is as defined in the first aspect. The preferred embodiments described for the first aspect extend these further aspects:
i) a pharmaceutical composition comprising a compound of formula (I), or a pharmaceutically acceptable derivative thereof, and a pharmaceutically acceptable carrier or diluent;
ii) the use of a compound of formula (I), or a pharmaceutically acceptable derivative thereof, in the manufacture of a medicament for the treatment or prophylaxis of a disorder characterised by the overexpression of TGF-β;
iii) the use of a compound of formula (I), or a pharmaceutically acceptable derivative thereof, in the manufacture of a medicament for the treatment or prophylaxis of a disorder mediated by the ALK5 receptor in mammals;
iv) the use of a compound of formula (I), or a pharmaceutically acceptable derivative thereof, in the manufacture of a medicament for the treatment or prophylaxis of a disorder selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids and restenosis;
v) the use of a compound of formula (I), or a pharmaceutically acceptable derivative thereof, in the manufacture of a medicament for the treatment or prophylaxis of kidney fibrosis;
vi) a compound of formula (I), or a pharmaceutically acceptable derivative thereof, for use as a medicament;
vii) a method of treatment or prophylaxis of a disorder selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids and restenosis, in mammals, which comprises administration to the mammal in need of such treatment, an effective amount of a compound of formula (I) or a pharmaceutically acceptable derivative thereof;
viii) a combination of a compound of formula (I) or a pharmaceutically acceptable derivative thereof, with an ACE inhibitor or an angiotensin II receptor antagonist; and
ix) a process for the preparation of a compound of formula (I) where two of X₁, X₂, and X₃ are N and the other is NH, comprising reacting a compound of formula (XVIII) 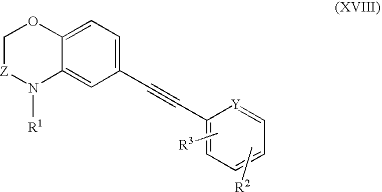
   with an azide source (preferably trimethylsilylazide) in a suitable solvent at a temperature greater than room temperature.

The pharmaceutically effective compounds of formula (I) and pharmaceutically acceptable salts thereof, may be administered in conventional dosage forms prepared by combining a compound of formula (I) with standard pharmaceutical carriers or diluents according to conventional procedures well known in the art. These procedures may involve mixing, granulating and compressing or dissolving the ingredients as appropriate to the desired preparation.

The following non-limiting examples illustrate the present invention.

### Intermediate 1

### (4-Bromo-2-nitrophenoxy)acetic acid ethyl ester

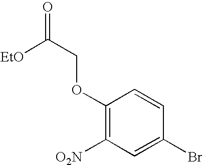

To a stirred solution of 4-bromo-2-nitrophenol (10 g, 45.87 mmol, 1.0 eq) in DMF (160 ml) at r.t. was added solid K₂CO3 (12.68 g, 91.74 mmol, 2.0 eq). The mixture was heated at 50° C. overnight, then allowed to cool to room temperature and partitioned between EtOAc and water. The organic phase was extracted with 1 N NaOH and water and dried over Na₂SO₄. The solvent was evaporated under reduced pressure to give a brown oil (5.01 g, 97%) which did not require further purification; ¹H NMR (350 MHz; CDCl3) δ: 7.85 (1H, d), 7.45 (1H, dd), 6.75 (1H, d), 4.55 (H, s), 4.05(2H, q), 1.10 (3H, t).

### Intermediate 2

### 6-Bromo-4H-benzo[1,4]oxazin-3-one

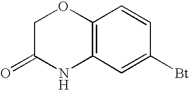

To a stirred solution of intermediate 1 (13.48 g, 44.33 mmol) in glacial, acetic acid (200 ml) was added iron powder (10eq, 24.6 g, 0.44 mol) at r.t. and the mixture was heated at 60° C. for two days. The mixture was filtered through a pan of celite and washed through with EtOAc. The filtrate was evaporated under reduced pressure and the residue was poured into a saturated solution of NaHCO₃ and extracted with ethyl acetate. The organic layer was washed with water, dried over Na₂SO₄ and filtered. The solvent was evaporated under reduced pressure to give the title compound as a white solid (8.2 g, 81%); m.p. 220° C.; ¹H NMR (350 MHz; CDCl₃) δ: 10.79 (1H, br.s), 7.09-7.01 (2H, m), 6.91 (1H, d), 4.59 (2H, s).

### Intermediate 3

### 6-Trimethylsilanylethynyl4H-benzo[1,4]oxazin-3-one

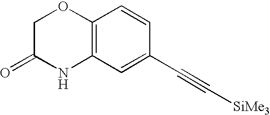

To a solution of intermediate 2 (2.02 g, 8.86 mmol) in dry THF (24 ml) under nitrogen were added TMEDA (24 ml) and TMS-acetylene (6 ml, excess). The resulting mixture was degassed with nitrogen, then tetrakis triphenylphosphine palladium (5% mol, 512 mg) and Cul (10% mol, 166 mg) were added. The resulting mixture was heated at 60° C. for 24 h. The reaction mixture was poured into a saturated solution of ammonium chloride and extracted with ethyl acetate. The organic phase was dried over Na₂SO₄ and filtered. Evaporation of the solvent in vacuo gave a crude product which was purified by chromatography on silica gel (petroleum ether/EtOAc 95:5 then 90:10). This gave the title compound (1.2 g, 55%) as a yellow solid; m.p. 142° C.; ¹H NMR (350 MHz; CDCl3) δ: 8.67 (1H, br.s), 7.12 (1H, dd), 6.93-6.85 (1H, m), 4.66 (2H, s), 0.26 (9H, s).

### Intermediate 4

### 6-Ethynyl-4H-benzo[1,4]oxazin-3-one

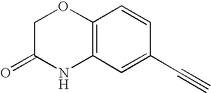

To a solution of Intermediate 3 (1 g, 4.07 mmol) in methanol (30 ml) was added potassium carbonate (3eq, 12.23 mmol, 1.69 g). The reaction mixture was then stirred at room temperature for one hour and the resulting suspension was filtered off. After concentration of the filtrate, the residue was dissolved in EtOAc and washed with water. The organic layer was dried over Na₂SO₄, filtered, and the solvent was evaporated under reduced pressure to give the title compound (795 mg, quantitative yield) as a white solid; ¹H NMR (350 MHz; CDCl₃) δ: 8.05 (1H, br.s), 6.97 (2H, dd), 6.78-6.75 (2H, m), 4.49(2H, s), 2.87 (1H, s).

### Intermediate 5

### 6-(6-Methyl-Dyridin-2-ylethynyl)-4H-benzo[1,4]oxazin-3-one

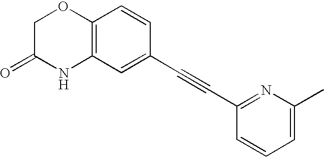

Intermediate 4 (705 mg, 4.07 mmol, 1 eq) and 2-bromo-6-methylpyridine (842 mg, 4.88 mmol, 1.2eq) were coupled and treated as described for intermediate 3 to afford the title compound as a yellow solid (930 mg, 86.5%) after chromatography on silica gel (CH₂Cl₂ then CH₂Cl₂/MeOH 99:1); ¹H NMR (300 MHz; CDCl₃) δ: 8.21 (1H, br.s), 7.42 (1H, t), 7.17 (1H, d), 7.07 (1H, dd), 6.96 (1H, d), 6.92 (1H, d), 6.79 (1H, d), 4.50(2H, s), 2.44 (3H, s); [MS APCl], m/z 265 (MH+).

### Intermediate 6

### 6-(Pyridin-2-ylethynyl)-4H-benzo[1,4]oxazin-3-one

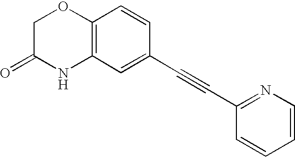

Intermediate 4 (705 mg, 4.07 mmol, 1eq) and 2-bromo-pyridine (771 mg, 4.88 mmol, 1.2eq) were coupled and treated as described for intermediate 3 to afford the title compound as a white solid (570 mg, 56%) after chromatography on silica gel (CH₂Cl₂ then CH₂Cl₂/MeOH 98:2); m.p. 210° C.; [MS APCl] m/z 251 (MH+).

### Intermediate 7

### 6-(3-Chloro-phenylethynyl)4H-benzo[1,4]oxazin-3-one

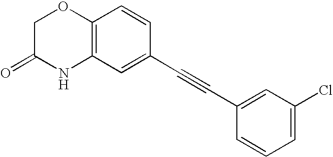

Intermediate 4 (6 g, 34.6 mmol) and 1-chloro-3-iodobenzene (5.15mL, 41.52 mmol, 1.2eq) were coupled and treated as described for intermediate 3 to afford the title compound as a yellow solid (5.84 g, 60%) after chromatography on silica gel (CH₂Cl₂ then CH₂Cl₂/MeOH 90:10); [MS APCl] m/z 282 MH−); ¹H NMR (300 MHz; DMSO-d₆) δ: 10.88 (1H, br.s), 7.64-7.61 (1H, m), 7.54-7.40 (3H, m), 7.14 (1H, dd), 7.03-6.98 (2H, m), 4.65 (2H, s).

### Intermediate 8

### 4-Methyl-6-(6-methyl-Dyridin-2-ylethynyl)-4H-benzo[1,4]oxazin-3-one

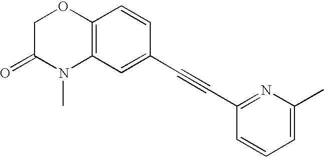

To a solution of intermediate 5 (460 mg, 1.74 mmol, 1 eq) in DMF (40 ml) was added K₂CO3(360 mg, 2.61 mmol) and iodomethane (370 mg, 2.61 mmol). The reaction mixture was stirred at room temperature for 1 hour and the resulting suspension was poured into water and extracted with EtOAc. The organic phase was dried over Na₂SO₄ and filtered. The solvent was removed under reduced pressure to afford the title compound as a red solid (0.4 g, 82.6%) which was used without purification in the next step; [MS APCl] m/z 279 (MH+).

### Intermediate 9

### 4-Ethyl-6-(6-methyl-pyridin-2-ylethynyl)-4H-benzo[1,4]oxazin-3-one

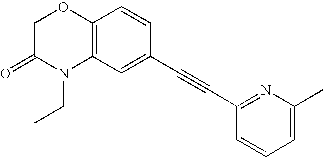

Intermediate 5 (264 mg, 1 mmol) and iodoethane (312 mg, 2eq) were coupled and treated as described for intermediate 8 to afford the title compound as an oil (292 mg, 100%); [MS APCl] m/z 293 (MH+).

### Intermediate 10

### 4-Propyl-6-(6-methyl-pyridin-2-ylethynyl)-4H-benzo[1,4]oxazin-3-one

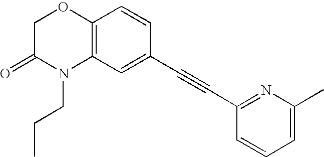

Intermediate 5 (700 mg, 2.65 mmol) and iodopropane (284 μL, 1.1eq) were coupled and treated as described for intermediate 8 to afford the title compound as a yellow oil (377 mg, 46.4%) after chromatography on silica gel (CH₂Cl₂/EtOAc 70:30 then 50/50); [MS APCl] m/z 307 (MH+).

### Intermediate 11

### 4-(Pronen-2-yl)-6-(6-methyl-pynidin-2-ylethynyl)4H-benzo[1,4]oxazin-3-one

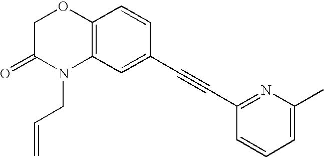

Intermediate 5 (786 mg, 2.97 mmol) and allylbromide (283 μL, 1.1eq) were coupled and treated as described for intermediate 8 to afford the title compound as an oil (565 mg, 62.5%) after chromatography on silica gel (CH₂Cl₂/EtOAc 90:10); [MS APCl] m/z 305 (MH+).

### Intermediate 12

### 4-(2-Methoxy-ethyl)-6-(6-methyl-pyridin-2-ylethynyl)-4H-benzo[1,4]oxazin-3-one

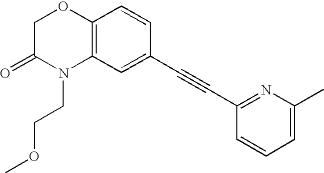

Intermediate 5 (528 mg, 2 mmol) and 2-bromoethyl methyl ether (417 mg, 1.5eq) were coupled and treated as described for intermediate 8 to afford the title compound as a yellow oil (600 mg, 93%); [MS APCl] m/z 323 (MH+).

### Intermediate 13

### 3-[6-(-6-Methyl-pyridin-2-ylethynyl)-3-oxo-2,3-dihydro-benzo[1,4]oxazin4-yl]-propionitrile

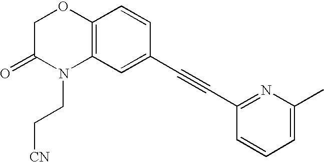

Intermediate 5 (700 mg, 2.65 mmol) and 3-bromo-propionitrile (242L, 1.1eq) were coupled and treated as described for intermediate 8 to afford the title compound as a beige solid (501 mg, 59.6%) after chromatography on silica gel (CH₂Cl₂/EtOAc 70:30 then 50/50); [MS APCl] m/z 318 (MH+).

### Intermediate 14

### 6-(6-Methyl-pyridin-2-ylethynyl)-4-(2-morpholin-4-yl-ethyl)4H-benzo[1,4]oxazin-3-one

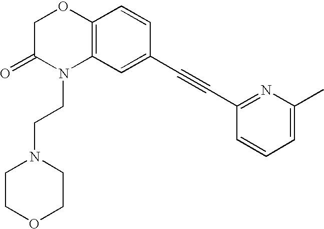

Intermediate 5 (528 mg, 2 mmol) and N-(2-chlorethyl)morpholine hydrochloride (558 mg, 1.5eq) were coupled and treated as described for intermediate 8 to afford the title compound as an oil (700 mg, 92%); [MS APCl] m/z 378 (MH+).

### Intermediate 15

### 4-(2-Dimethylamino-ethyl)-6-(6-methyl-pyridin-2-ylethynyl)-4H-benzo[1,4]oxazin-3-one

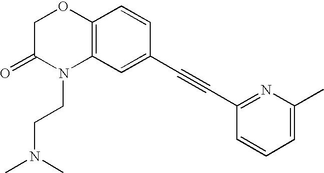

Intermediate 5 (700 mg, 2.65 mmol) and 2-dimethylaminoethyl chloride hydrochloride (382 mg, 1.1 eq) were coupled and treated as described for intermediate 8 to afford the title compound as a yellow oil (401 mg, 45%) after chromatography on silica gel (CH₂Cl₂/EtOAc 70:30 then 50/50); [MS APCl] m/z 336 (MH+).

### Intermediate 16

### 4-Ethyl-6-(6-methyl-pyridin-2-ylethynyl)-3,4dihydro-2H-benzo[1,4]oxazine

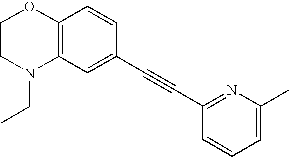

To a solution of intermediate 9 (675 mg, 2.3 mmol) in anhydrous THF (100 ml) under nitrogen, was added a solution of 0.5M 9-BBN in THF (10.15mL, 9.4eq,). The reaction mixture was stirred at 60° C. overnight. The reaction was hydrolysed with wet Na₂SO₄. The mixture was filtered and the solvent was evaporated under reduced pressure. The crude product was treated with 1 N HCl, washed with diethyl ether and basified with triethylamine. The aqueous phase was extracted with EtOAc and the organic phases were combined and dried over Na₂SO4. Evaporation of the solvent gave a yellow oil (263 mg, 41%); 1H NMR (300 MHz; CDCl3) δ: 7.45 (1H, t), 7.25 (1H, d), 7.00 (1H, d), 6.85 (1H, s), 6.80 (1H, d), 6.65 (1H, d), 4.25-4.15 (2H, m), 3.32-3.02 (4H, m), 2.45 (3H, s), 1.05 (3H, t), triazole NH not observed; [MS APCl] m/z 279 (MH+).

### Intermediate 17

### 4-Methyl-6-(6-methyl-pyridin-2-ylethynyl)-3,4-dihydro-2H-benzo[1,4]oxazine

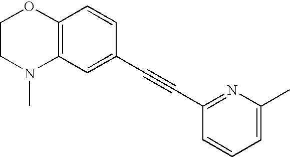

Intermediate 8 was treated with 9-BBN as described for intermediate 15, to give the title compound as a yellow oil (1.28 g, 76.26%); ¹H NMR (300 MHz; CDCl3) δ: 7.39 (1H, t), 7.17 (1H, d), 7.00 (1H, d), 6.92 (1H, d), 6.80-6.74 (2H, m), 6.57 (1H, d), 4.16-4.14 (2H, m), 3.14-3.08 (2H, m), 2.73 (3H, s), 2.42 (3H, s), triazole NH not observed; [MS APCl] m/z 265 (MH+).

### Intermediate 18

### 1-[6-Trimethylsilanylethynyl-2,3-dihydro-benzo[1,4]oxazin-4-yl]ethanone

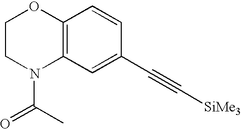

The title compound was obtained as a red oil from 4-acetyl-3,4-dihydro-2H-benzo[b][1,4]oxazin-6-yl bromide (5 g, 19.52 mmol, 1eq) according to the procedure described for intermediate 3 (4.54 g, 85%); ¹H NMR (300 MHz; CDCl₃) δ: 7.22 (1H, d), 6.84 (1H, d), 4.33-4.30 (2H, s), 3.94 (2H, m), 2.36 (3H,s), 0.25 (9H, s).

### Intermediate 19

### 1-[6-Ethynyl-2,3-dihydro-benzo[1,4]oxazin4-yl]ethanone

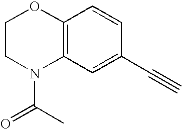

The title compound was obtained as a brown oil from intermediate 18 (4.5 g, 16.46 mmol, 1eq) according to the procedure described for Intermediate 4 (1.54 g, 49.4%) in mixture with 6-ethynyl-3,4-dihydro-2H-benzo[1,4]oxazine (1.19, 42%); ¹H NMR (300 MHz; CDCl₃) δ: 7.23 (1H, d), 6.87 (1H, d), 4.32 (2H, t), 4.00-3.90 (2H, m), 3.02 (1H,s), 2.35 (3H, s).

### Intermediate 20

### 4-Acetyl-6-(6-methyl-pyridin-2-ylethynyl)-2,3-dihydro-2H-benzo[1,4]-oxazine

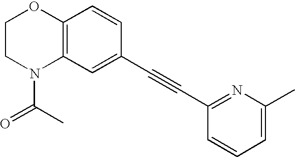

Intermediate 19 (1.54 g, 8.14 mmol, 1eq) and 2-bromo-6-methylpyridine (1.68 g, 9.77 mmol, 1.2eq) were coupled and treated as described for intermediate 3 to afford the title compound as a yellow oil (1.80 g, 75.6%) after chromatography on silica gel (CH₂Cl₂ then CH₂Cl₂/MeOH 99: 1); ¹H NMR (300 MHz; CDCl₃) δ: 7.61-7.56 (1H, m), 7.34 (2H,, d), 7.12 (1H, d), 6.90 (1H, d), 4.33 (2H, t), 4.00-3.94 (2H, m), 2.59 (3H,s), 2.36(3H, s); [MS APCl] m/z 293 (MH+).

### Intermediate 21

### 6-(6-Methyl-pyridin-2-ylethynyl)-2.3-dihydro-2H-benzo[1,4]-oxazine

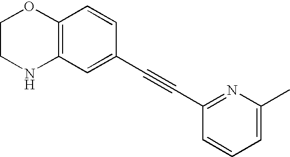

To a solution of intermediate 20 (0.612 g, 2.09 mmol, 1eq) in methanol (20 ml) was added solid K₂CO₃(1.16 g, 8.36 mmol, 4 eq). The mixture was heated at 50° C. for 1 h. To complete the reaction four more equivalents of K₂CO₃ (1.16 g ) were added and the reaction mixture was heated at 65° C. for 3 h. The mixture was concentrated under reduced pressure and partitioned between EtOAc and water. The organic phase was washed with a saturated solution of NaCl and dried over Na₂SO₄. The solvent was evaporated under reduced pressure to give a colourless oil (0.476 g, 91%) which did not require further purification; [MS APCl] m/z 251 (MH+).

### Intermediate 22

### 4-(2-Propenyl)6-(6-methyl-pyridin-2-ylethynyl)-2.3-dihydro-2H-benzo[1,4]-oxazine

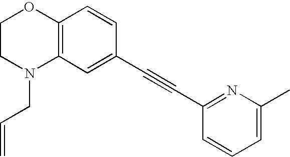

To a solution of intermediate 21 (438 mg, 0.95 mmol, 1 eq) in DMF (20m1) was added K₂CO₃(263 mg, 2.61 mmol, 2eq) and allylbromide (99 μl, 1.14 mmol, 1.2eq). The reaction mixture was stirred at 60° C. for 4 h. Further K₂CO₃ (1 eq) and allylbromide (2 eq) were added and the reaction mixture was stirred at 60° C. for 48 h. The solvent was evaporated to dryness and the resulting solid was partitioned between water and EtOAc. The organic phase was dried over Na₂SO₄ and filtered. The solvent was removed under reduced pressure to afford the title compound as a yellow oil (430 mg, quantitative) which was used without purification in the next step; [MS APCl] m/z 291 (MH+).

### Intermediate 23

### 4-(Chloroacetyl)-6-(6-methyl-pyridin-2-ylethynyl)-2,3-dihydro-2H-benzo[1,4]-oxazine

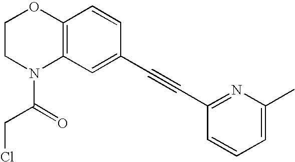

To a solution of intermediate 21 (2.7 g, 10.8 mmol, 1eq) in dichloromethane (40 ml) was added triethylamine (1.41 g, 14 mmol, 1.3eq) and chloroacetyl chloride (1.58 g, 14 mmol, 1.3eq). The reaction mixture was stirred at room temperature overnight. The reaction mixture was poured into water and extracted with CH₂Cl₂. The organic phase was dried over Na₂SO₄ and filtered. The solvent was removed under reduced pressure to afford the title compound as a brown oil (3 g, 85%) which was used without purification in the next step; [MS APCl] m/z 327 (MH+).

### Intermediate 24

### 44(4-Methyl-1-piperazinyl)acetyl-6-(6-methyl-pyridin-2-ylethynyl)-2,3-dihydro-2H-benzo[1,4]-oxazine

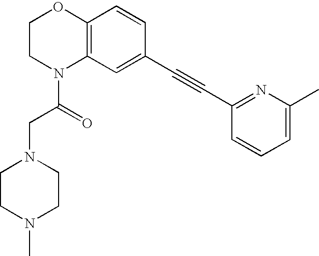

To a solution of intermediate 23 (326 mg, 1 mmol, 1 eq) in acetone (20mi) was added K₂CO₃ (414 mg, 3 mmol, 3eq) and 1-methylpiperazine (503 mg, 5 mmol, 5eq). The mixture was heated at reflux overnight. The reaction mixture was filtered and evaporated to dryness. The residue was suspended in water and extracted with EtOAc. The organic phase was dried over Na₂SO₄ and filtered. The solvent was removed under reduced pressure to afford the title compound as a brown oil (250 mg, 64%) which was used without purification in the next step; [MS APCl] m/z 391 (MH+).

### EXAMPLE 1

### 6-[5-(6-Methyl-pyridin-2-yl)-1H-[1,2,3]triazol-4-yl]-4H-benzo[1,4]oxazin-3-one

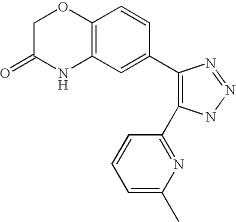

To a solution of Intermediate 5 (500 mg, 1.89 mmol) in dry DMF (8 ml) was added azidotrimethylsilane (4eq, 7.56 mmol, 870 mg). The reaction mixture was then stirred at 100° C. for two days. The reaction mixture was allowed to cool and the solvent was removed by distillation under reduced pressure. The residue was partitioned between water and EtOAc and the layers separated. The organic phase was dried over Na₂SO₄ and filtered. Evaporation of the solvent in vacuo gave a crude product which was purified by chromatography on silica gel (CH₂Cl₂/MeOH 95:5+0.1 %Et3N) to give the title compound (200 mg, 34.4%) as an off-white solid after recrystallisation in EtOH/petroleum ether; mp. 224° C.; [MS APCl] m/z 308.2 (MH+); ¹H NMR (300 MHz; DMSO-d⁶) δ: 10.58 (1H, br.s), 7.56 (1H, t), 7.36-7.05 (4H, br.m), 6.76 (1H, br.d), 4.41 (2H, s), 2.28 (3H, s), triazole NH not observed.

### EXAMPLE 2

### 6-(5-Pyridin-2-yl)-1H-[1,2,3]triazol-4-yl-4H-benzo[1,4]oxazin-3-one

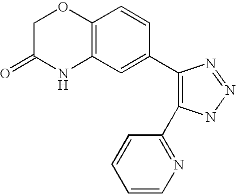

Intermediate 6 and azidotrimethylsilane were reacted as described for example 1. to give the title compound as a white solid (230 mg, 35%); m.p. 256° C.; [MS APCl] m/z 294 (MH+); ¹H NMR (300 MHz; DMSO-d⁶) δ: 10.72 (1H, s), 8.53 (1H, d), 7.82 (1H, td), 7.69 (1H, d), 7.35-7.26 (2H, m), 7.16 (1H, dd), 6.89 (1H, d), 4.54 (2H, s), triazole NH not observed.

### EXAMPLE 3

### 6-r5-(3-Chloro-phenyl)-1H-[1,2,3]triazol4-yl-4H-benzo[1,4]oxazin-one

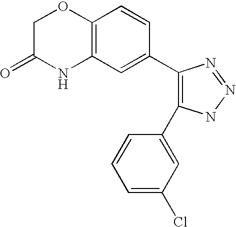

Intermediate 7 and azidotrimethylsilane were reacted as described for example 1 to give the title compound as a white solid (142 mg, 17.6%); m.p. 220° C.; ¹H NMR (300 MHz; DMSO-d⁶) δ: 10.76 (1H, br.s), 7.55-7.35 (4H, m), 7.13-6.87 (3H,m), 4.58 (2H, s), triazole NH not observed; TOF MS ES⁺ exact mass calculated for C₁₋₆H₁₁ClN₄0₂ (MH+):327.0649. Found: 327.0667.

### EXAMPLE 4

### 4-Methyl-6-[5-(6-methyl-pyridin-2-yl)-1H-[1,2,3]triazol-4-yl]-4H-benzo[1,4]oxazin-3-one

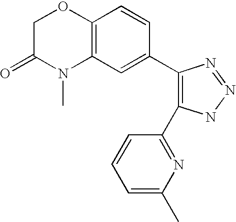

Intermediate 8 and azidotrimethylsilane were reacted as described for example 1 to give the title compound as a beige solid (290 mg, 64.5%); ¹H NMR (300 MHz; DMSO-d⁶) δ: 7.88 (1H, t), 7.72-7.66 (2H, m), 7.53 (1H, dd), 7.38 (1H, d), 7.15 (1H,d), 4.80 (2H, s), 3.35(3H, s), 2.58 (3H, s), triazole NH not observed; TOF MS ES⁺ exact mass calculated for C₁₇H₁₅N₅O₂ (MH+):322.1304. Found: 322.1306.

### EXAMPLE 5

### 4-Ethyl-6-[5-(6-methyl-pyridin-2-yl)-1H-[1,2,3]triazol4-yl]-4H-benzo[1,4]oxazin-3-one

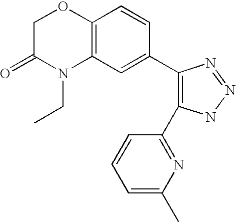

Intermediate 9 and azidotrimethylsilane were reacted as described for example 1 to give the title compound as a white solid (155 mg, 46%); m.p. 187° C.; ¹H NMR (300 MHz; CDCl₃) 8: 7.64 (1H, t), 7.46 (1H, d), 7.39-7.30 (2H, m), 7.20 (1H, d), 7.04 (1H, d), 4.87 (2H, s), 3.97 (2H, q), 2.63 (3H, s), 1.21 (3H, t), triazole NH not observed; TOF MS ES⁺ exact mass calculated for C₁₈H₁₇N₅O₂ (MH+):336.1,460. Found: 336.1431.

### EXAMPLE 6

### 4-Propyl-6-[5-(6-methyl-pyridin-2-yl)-1H-[1,2,3]triazol-4-yl]4H-benzo[1,4]oxazin-3-one

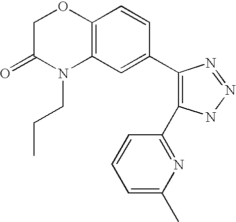

Intermediate 10 and azidotrimethylsilane were reacted as described for example 1 to give the title compound as a yellow solid (240 mg, %), gummy at 100° C.; ¹H NMR (300 MHz; DMSO-d₆) δ: 7.59 (1H, t), 7.40-6.99 (4H, m), 6.87 (1H, d), 4.48 (2H, s), 3.59-3.50 (2H, m), 2.26 (3H, s), 1.30-1.17 (2H, m), 0.55 (3H, t), triazole NH not observed; TOF MS ES⁺ exact mass calculated for C₁₉H₁₉N₅O₂ (MH+):350.1617. Found: 350.1602.

### EXAMPLE 7

### 4-(Propen-2-yl)-6-r5-(6-methyl-pyridin-2-yl)-1H-[1,2,3triazol4-yl]-4H-benzo[1,4]oxazin-3-one

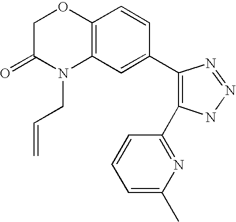

Intermediate 11 and azidotrimethylsilane were reacted as described for example 1 to give the title compound as a beige solid (435 mg, 67%), gummy at 120° C.; ¹H NMR (300 MHz; DMSO-d₆) δ: 7.94 (1H, t), 7.73-7.41 (4H, m), 7.23 (1H, d), 5.98-5.83 (1H, m), 5.25-5.09 (2H, m), 4.92 (2H, s), 4.59 (1H, br s), 2.64 (3H, s), triazole NH not observed; TOF MS ES⁺ exact mass calculated for C₁₉H₁₇N₅0₂ (MH+):348.1460. Found: 348.1445

### EXAMPLE 8

### 4-(2-Methoxy-ethyl)6-[5-(6-methyl-pyridin-2-yl)-1H-[1,2,3]triazol-4-yl]-4H-benzo[1,4]oxazin-3-one

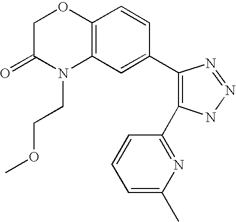

Intermediate 12 and azidotrimethylsilane were reacted as described for example 1 to give the title compound as a yellow solid (294 mg, 44%); ¹H NMR (300 MHz; CDCl₃) δ: 7.61 (1H, t), 7.53 (1H, s), 7.43 (1H, d), 7.32 (1H, d), 7.16 (1H, d), 7.00 (1H, d), 4.65 (2H, s), 4.09 (2H, t), 3.58 (2H, t), 3.27 (3H, s), 2.60 (3H, s), triazole NH not observed; TOF MS ES⁺ exact mass calculated for C₁₉H₁₉N₅O₃ (MH+):366.1566. Found: 366.1552.

### EXAMPLE 9

### 3-{6-[5-(-6-Methyl-pyridin-2-yl)-1H-[1,2,3]triazol4-yl]-3-oxo-2,3-dihydro-benzo[1,4]oxazin-4-yl}-propionitrile

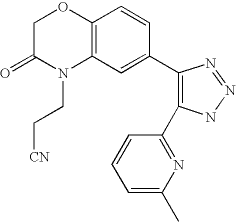

Intermediate 13 and azidotrimethylsilane were reacted as described for example I to give the title compound as an orange powder (310 mg, 54.4%); ¹H NMR (300 MHz; DMSO-d₆) δ: 7.98 (1H, t), 7.83-7.61 (3H, m), 7.47 (1H,d), 7.30 (1H,d), 4.94 (2H, s), 4.38 (2H, br t), 3.06-2.96 (2H, m), 2.67 (3H,s), triazole NH not observed; TOF MS ES⁺ exact mass calculated for C₁₉H₁₆N₆O₂ (MH+):361.1413. Found: 361.1431.

### EXAMPLE 10

### 6-[5-(6-Methyl-pyridin-2-yl)-1H-[1,2,3]triazol4yl]-4-(2-morpholin-4-yl-ethyl)-4H-benzo[1,4]oxazin-3-one

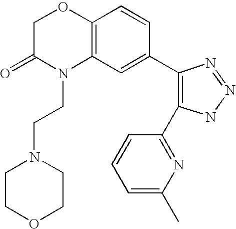

Intermediate 14 and azidotrimethylsilane were reacted as described for example 1 to give the title compound as a yellow solid (340 mg, 45%); m.p. 150° C.; ¹H NMR (300 MHz; CDCl₃) δ: 7.56 (1H, t), 7.43-7.33 (2H, m), 7.24 (1H, dd), 7.10(1H, d), 6.90 (1H, d), 4.56 (2H, s), 4.04 (2H, br t), 3.67-3.57 (4H, m), 2.57-2.40 (9H, m), triazole NH not observed; TOF MS ES⁺ exact mass calculated for C22H₂₄N6O₃ (MH+):421.1988. Found: 421.2002.

### EXAMPLE 11

### 4-(2-Dimethylaminoethyl)-6-[5-(6-methyl-Dyridin-2-yl)-1H-[1,2,3]triazol-4-yl]-4H-benzo[1,4]oxazin-3-one

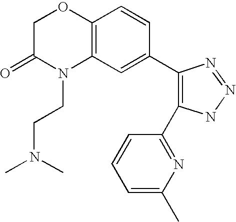

Intermediate 15 and azidotrimethylsilane were reacted as described for example 1 to give the title compound as an off-white powder (133 mg, 29.3%), gummy at 100° C.; ¹H NMR (300 MHz; DMSO-d6) δ: 7.80 (1H, t), 7.62-7.42 (3H, m), 7.32 (1H, d), 7.11 (1H, d), 4.73 (2H, s), 3.96 (2H, br t), 2.51 (3H, s), 2.33 (2H, br t), triazole NH not observed; TOF MS ES⁺ exact mass calculated for C₂₀H₂₂N₆O₂ (MH+):379.1882. Found (H.R.M.S): 379.1886.

### EXAMPLE 12

### 4-Ethyl-6-[5-(6-methyl-pyridin-2-yl)-1H-[1,2,3]triazol-4-yl]-3,4-dihydro-2H-benzo[1,4]oxazine

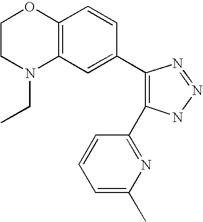

Intermediate 16 and azidotrimethylsilane were reacted as described for example 1 to give the title compound as a beige solid (10 mg, 3.3%); ¹H NMR (300 MHz; CDCl3) δ: 7.50 (1H, t), 7.38 (1H, d), 7.05 (1H, d), 6.90 (1H, s), 6.80 (1H, d), 6.70 (1H, d), 4.25-4.18 (2H, m), 3.30-3.15 (4H, m), 2.50 (3H, s), 1.00 (3H, t), triazole NH not observed; TOF MS ES⁺exact mass calculated for C₁₈H₁₉N₅O (MH+):322.1668. Found: 322.1666.

### EXAMPLE 13

### 4-Methyl-6-[5-(6-methyl-pyridin-2-yl)-1H-[1,2,3]triazol-4-yl]-3,4-dihydro-2H-benzo[1,4]oxazine

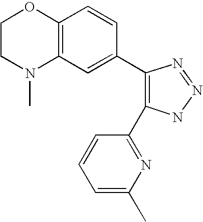

Intermediate 17 and azidotrimethylsilane were reacted as described for example 1 to give the title compound as a pale yellow powder (220 mg, 16.8%); ¹H NMR (300 MHz; CDCl3) δ: 7.52-7.37 (2H, m), 7.05 (1H, d), 6.95 (1H, d), 6.84 (1H, dd), 6.72 (1H, d), 4.294.26 (2H, m), 3.23-3.21 (2H, m), 2.78 (3H, s), 2.52 (3H, s), triazole NH not observed; TOF MS ES⁺ exact mass calculated for C₁₇H₁₇N₅O (MH+):308.151 1. Found: 308.1511.

### EXAMPLE 14

### 4-Acetyl-6-[5-(6-methyl-pyridin-2-yl)-1H-[1,2,3]-triazol-4-yl]-3.4-dihydro-2H-benzo[1,4]-oxazine

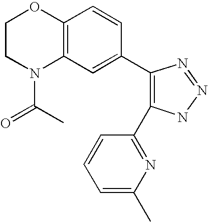

Intermediate 20 and azidotrimethylsilane were reacted as described for example 1 to give the title compound as an off-white powder (690 mg, 33.6%); ¹H NMR (300 MHz; DMSO-d₆) δ: 7.88 (1H, t), 7.67 (1H, d), 7.56 (1H, d), 7.37 (1H, d), 7.06 (1H, d), 4.44-4.41 (2H, m), 4.01-3.98(2H, m), 2.58 (3H, s), 2.30 (3H, s), triazole NH not observed; TOF MS ES⁺ exact mass calculated for C1₈H₁₇N₅0₂ (MH+):336.1460. Found: 336.1458.

### EXAMPLE 15

### 6-[5-(6-methyl-pyridin-2-yl)1H-[1,2,3]-triazol]-3,4-dihydro-2H-benzo[1,4]-oxazine

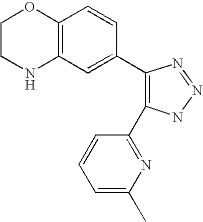

To a solution of example 14 (335 mg, 0.9 mmol, 1eq) in a mixture of MeOH (5 ml) and water (3 ml) was added KOH (360 mg, 6.41 mmol, 4 eq). The mixture was heated at 60° C. for 18 h. The mixture was concentrated under reduced pressure. The residue was diluted in water, neutralised with 1 N HCl and extracted with EtOAc. The organic phase was dried over Na₂SO₄, filtered and concentrated under reduced pressure. The crude product was purified by chromatography on silica gel (CH₂Cl₂/MeOH 99:1 then 95:5) to give an oil which was organised in pentane to afford the title compound as a yellow powder (120 mg, 41%); 1H NMR (300 MHz; CDCl₃) δ: 7.54-7.35 (2H, m), 7.05 (1H, d), 6.93-6.69 (3H, m), 4.26-4.20 (2H, m), 3.41-3.35 (2H, m), 2.53 (3H, s), triazole NH not observed; TOF MS ES⁺ exact mass calculated for C₁₆H₁₅N₅O (MH+): 294.1355. Found: 294.1387.

### EXAMPLE 16

### 4-(Propen-2-yl)-6-[5-(6-methyl-pyridin-2-yl)-1H-[1,2,3]-triazol-4-yl]-3,4-dihydro-2H-benzo[1,4]-oxazine

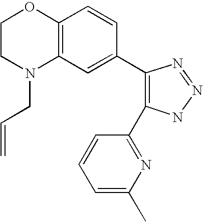

Intermediate 22 and azidotrimethylsilane were reacted as described for example 1 to give the title compound as an orange solid (39 mg, 8%), gummy at 120° C.; ¹H NMR (300 MHz; CDCl3) δ: 7.68-7.50 (2H, m), 7.20 (1H, d), 7.09-6.86 (3H, m), 5.93-5.78 (1H, m), 5.31-5.16 (2H, m), 4.42-4.34 (2H, m), 3.93-3.86 (2H, br d), 3.46-3.39 (2H, m), 2.68 (3H, s), triazole NH not observed; TOF MS ES⁺ exact mass calculated for C₁₉H₁₉N₅O (MH+):334.1668. Found: 334.11643.

### EXAMPLE 17

### 4-[(4-Methyl-1-piperazinyl)acetyl]-6-(6-methyl-pyridin-2-yl-1H-[1,2,3]-triazol-4-yl)-3.4-dihydro-2H-benzo[1,4]-oxazine

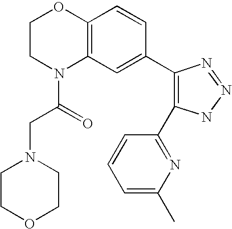

Intermediate 24 and azidotrimethylsilane were reacted as described for example 1 to give the title compound as an off-white solid (24 mg, 8.7%), m.p. 132° C.; TOF MS ES⁺ exact mass calculated for C23H₂₇N₇O₂ (MH+):434.2304. Found : 434.2315.

Biology

The biological activity of the compounds of the invention may be assessed using the following assays:

Assay 1 (Cellular Transcriptional Assay)

The potential for compounds of the invention to inhibit TGF-β signaling may be demonstrated, for example, using the following in vitro assay.

The assay was performed in HepG2 cells stably transfected with the PAl-1 promoter (known to be a strong TGF-β responsive promoter) linked to a luciferase (firefly) reporter gene. The compounds were selected on their ability to inhibit luciferase activity in cells exposed to TGF-β. In addition, cells were transfected with a second luciferase (Renilla) gene which was not driven by a TGF-β responsive promoter and was used as a toxicity control.

96 well microplates were seeded, using a multidrop apparatus, with the stably transfected cell line at a concentration of 35000 cells per well in 200 μl of serum-containing medium. These plates were placed in a cell incubator.

18 to 24 hours later (Day 2), cell-incubation procedure was launched. Cells were incubated with TGF-β and a candidate compound at concentrations in the range 50 nM to 10 μM (final concentration of DMSO 1%). The final concentration of TGF-β (rhTGFβ-1) used in the test was 1 ng/mL. Cells were incubated with a candidate compound 15-30 mins prior to the addition of TGF-β. The final volume of the test reaction was 150 μl. Each well contained only one candidate compound and its effect on the PAl-1 promoter was monitored.

Columns 11 and 12 were employed as controls. Column 11 contained 8 wells in which the cells were incubated in the presence of TGF-β, without a candidate compound. Column 11 was used to determine the ‘reference TGF-β induced firefly luciferase value’ against which values measured in the test wells (to quantify inhibitory activity) were compared. In wells A12 to D12, cells were grown in medium without TGF-β. The firefly luciferase values obtained from these positions are representative of the ‘basal firefly luciferase activity’. In wells E12 to H12, cells were incubated in the presence of TGF-β and 500 μM CPO (Cyclopentenone, Sigma), a cell toxic compound. The toxicity was revealed by decreased firefly and renilla luciferase activities (around 50 % of those obtained in column 11).

12 to 18 hours later (day 3), the luciferase quantification procedure was launched. The following reactions were performed using reagents obtained from a Dual Luciferase Assay Kit (Promega). Cells were washed and lysed with the addition of 10 μl of passive lysis buffer (Promega). Following agitation (15 to 30 mins), luciferase activities of the plates were read in a dual-injector luminometer (BMG lumistar). For this purpose, 50 μl of luciferase assay reagent and 50 μl of ‘Stop & Glo’ buffer were injected sequentially to quantify the activities of both luciferases. Data obtained from, the measurements were processed and analysed using suitable software. The mean Luciferase activity value obtained in wells A11 to H11 (Column 11, TGF-βonly) was considered to represent 100% and values obtained in wells A12 to D12 (cells in medium alone) gave a basal level (0%). For each of the compounds tested, a concentration response curve was constructed from which an IC₅₀ value was determined graphically.

Assay 2 (Alk5 Fluorescence Polarization Assay)

Kinase inhibitor compounds conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This. protocol details the use of a rhodamine green-labelled ligand for assays using recombinant GST-ALK5 (residues 198-503).

Assay buffer components: 62.5 mM Hepes pH 7.5 (Sigma H4034), 1 mM DTT (Sigma D-0632), 12.5 mM MgCl₂ (Sigma M-9272), 1.25 mM CHAPS (Sigma C-3023). Protocol: Solid compound stocks were dissolved in 100% DMSO to a concentration of 1 mM and transferred into column 1, rows A-H of a 96-well, U bottom, polypropylene plate (Costar #3365) to make a compound plate. The compounds were serially diluted (3-fold in 100% DMSO) across the plate to column 11 to yield 11 concentrations for each test compound. Column 12 contained only DMSO. A Rapidplate™-96 was used to transfer 1 μl of sample from each well into a 96-well, black, U-bottom, non-treated plate (Costar #3792) to create an assay plate. ALK5 was added to assay buffer containing the above components and 1 nM of the rhodamine green-labelled ligand so that the final ALK5 concentration was 10 nM based on active site titration of the enzyme. The enzyme/ligand reagent (39 μl) was added to each well of the previously prepared assay plates. A control compound (1 μl) was added to column 12, rows E-H for the low control values. The plates were read immediately on a UL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest reader and then imported into curve fitting software for construction of concentration response curves. The normalized response was determined relative to the high controls (1 μl DMSO in column 12, rows A-D) and the low controls (1 μl of control compound in column 12, rows E-H). An IC₅₀ value was then calculated for each compound

The compounds of this invention generally show ALK5 receptor modulator activity having IC₅₀ values in the range of 1 to 100 nM and TGF-β cellular activity having IC₅₀ values in the range of 0.001 to 10 μM

The application of which this description and claims forms part may be used as a basis for priority in respect of any subsequent application. The claims of such subsequent application may be directed to any novel feature or combination of features described herein. They may take the form of product, composition, process or use claims and may include, by way of example and without limitation, the following claims:

1. A compound of formula (i), or a pharmaceutically acceptable derivative thereof: 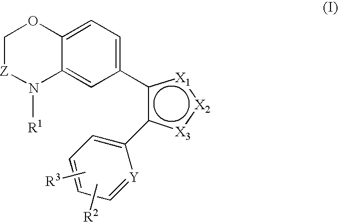
wherein Z is CH₂ or C═O;
Y is N or CH;
R¹ is selected from H, C₁₋₆alkyl, C₂₋₆alkenyl, —(CH₂)ₚ—NR⁴R⁵, —(CH₂)ₚ—OR⁴, —(CH₂)ₚ—CN, —(CH₂)ₚ—CONR⁴R⁵, —(CH₂),—NHCOR⁴, —(CH₂)ₚ—NHSO₂R⁴ and —(CH₂)ₚ-het, wherein the het group is optionally substituted by C₁₋₆alkyl; or when Z is CH₂, R¹ may additionally be selected from —CO-C₁₆alkyl, —CO—(CH₂)_{q}—OR⁴, —CO—(CH₂)_{q}—NR⁴R⁵ and —CO—(CH₂)_{q}-het wherein the het group is optionally substituted by C, ₆alkyl;
R² is selected from H, C₁₋₆alkyl, halo, CN or perfluoroC,6alkyl;
R³ is selected from H or halo;
R⁴ and R⁵ are independently selected from H or C₁₋₆alkyl; or R⁴ and R⁵ together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₄ alkyl and C₁₋₄ alkoxy;
two of X₁, X₂ and X₃ are N and the other is NR⁶ wherein R⁶ is hydrogen, C₁₋₆alkyl, C₃₋₇cycloalkyl, —(CH₂)ₚ—CN, —(CH₂)ₚ—CO₂H, —(CH₂), —CONR⁷R⁸, —(CH₂)ₚCOR⁷, —(CH2)_{q}(OR⁹)2, —(CH₂)ₚOR⁷, —(CH2)_{q}—CH═CH—CN, —(CH₂)_{q}—CH═CH—CO₂H, —(CH₂)_{q}—CH═CH—CONR⁷R⁸, (CH₂)ₚNHCOR¹⁰ or —(CH₂)ₚNR¹¹R¹²;
R⁷ and R⁸ are independently H, C, ₆alkyl, aryl or het;
R⁹ is C₁₆alkyl;
R¹⁰ is C₁₋₇alkyl, aryl, heteroaryl, arylC₁₋₆alkyl or heteroarylC₁₋₆alkyl;
R¹¹ and R¹² are independently selected from hydrogen, C₁₋₆alkyl, aryl and arylC₁₋₆alkyl, or R¹¹ and R¹² together with the atom to which they are attached form a 3, 4, 5, 6 or 7 membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₄ alkyl and C₁₋₄ alkoxy;
p is 2-4; and
q is 1-4.

2. A compound according to claim 1, or a pharmaceutically acceptable derivative thereof, wherein Y is N.

3. A compound according to claim 1, or a pharmaceutically acceptable derivative thereof, wherein R¹ is H, C₁₋₆alkyl, C₂₋₆alkenyl, —(CH₂)₂-Het, —(CH₂)₂—OR⁴, —(CH₂)₂—NR⁴R⁵, or —(CH₂)₂—CN.

4. A compound according to claim 3, or a pharmaceutically acceptable derivative thereof, wherein R¹ is H, C₁₋₆alkyl or C₂₋₆alkenyl.

5. A compound according to claim 1, or a pharmaceutically acceptable derivative thereof, wherein R² is H, C₁₋₆alkyl or halo.

6. A compound according to claim 5, or a pharmaceutically acceptable derivative thereof, wherein when Y is N, R² is methyl positioned ortho to Y.

7. A compound according to claim 1, or a pharmaceutically acceptable derivative thereof, wherein R³ is H or halo.

8. A compound according to claim 7, or a pharmaceutically acceptable derivative thereof, wherein when Y is N and R² is methyl positioned ortho to Y, R³ is H.

9. A compound according to claim 1, or a pharmaceutically acceptable derivative thereof, wherein R⁶ is H.

10. A compound according to claim 1 selected from:
6-[5-(6-methyl-pyridin-2-yl)-1H-[1,2,3]triazol-4-yl]-4H-benzo[1,4]oxazin-3-one (Example 1);
6-(5-pyridin-2-yl-1H-[1,2,3]triazol-4-yl)-4H-benzo[1,4]oxazin-3-one (Example 2);
6-[5-(3-chloro-phenyl)-1H-[1,2,3]triazol-4-yl]-4H-benzo[1,4]oxazin-3-one (Example 3);
4-methyl-6-[5-(6-methyl-pyridin-2-yl)-1H-[1,2,3]triazol-4-yl]-4H-benzo[1,4]oxazin-3-one (Example 4);
4-ethyl-6-[5-(6-methyl-pyridin-2-yl)-1H-[1,2,3]triazol-4-yl] -4H-benzo[1,4]oxazin-3-one (Example 5);
4-propyl-6-[5-(6-methyl-pyridin-2-yl)-1H-[1,2,3]triazol-4-yl]-4H-benzo[1,4]oxazin-3-one (Example 6);
4-(propen-2-yl)-6-[5-(6-methyl-pyridin-2-yl)-1H-[1,2,3]triazol-4-yl]-4H-benzo[1,4]oxazin-3-one (Example 7);
4-(2-methoxy-ethyl)-6-[5-(6-Methyl-pyridin-2-yl)-1H-[1,2,3]triazol-4-yl]-4H-benzo[1,4]oxazin-3-one (Example 8);
3-{6-[5-(-6-methyl-pyridin-3-yl)-1H-[1,2,3]triazol-4-yl]-3-oxo-2,3-dihydro-benzo[1,4]oxazin-4-yl}-propionitrile (Example 9);
6-[5-(6-methyl-pyridin-2-yl)-1H-[1,2,3]triazol-4-yl]-4-(2-morpholin-4-yl-ethyl)-4H-benzo[1,4]oxazin-3-one (Example 10);
4-(2-dimethylamino-ethyl)-6-[5-(6-Methyl-pyridin-2-yl)-1H-[1,2,3]triazol-4-yl]-4-(2-morpholin-4-yl-ethyl)-4H-benzo[1,4]oxazin-3-one (Example 11);
6-[5-(6-methyl-pyridin-2-yl)-1H-[1,2,3]-triazol-4-yl]-3,4-dihydro-2H-. benzo[1,4]-oxazine (Example 15);
4-methyl-6-[5-(6-methyl-pyridin-2-yl)-1H-[1,2,3]-triazol-4-yl]-3,4-dihydro-2H-benzo[1,4]-oxazine (Example 13);
4-acetyl-6-[5-(6-methyl-pyridin-2-yl)-1H-[1,2,3]-triazol-4-yl]-3,4-dihydro-2H-benzo[1,4]-oxazine (Example 14);
4-ethyl-6-[5-(6-methyl-pyridin-2-yl)-1H-[1,2,3]-triazol-4-yl]-3,4-dihydro-2H-benzo[1,4]-oxazine (Example 12);
4-(propen-2-yl)-6-[5-(6-methyl-pyridin-2-yl)-1H-[1,2,3]-triazol-4-yl]-3,4-dihydro-2H-benzo[1,4]-oxazine (Example 16); and
4-[(4-methyl-1-piperazinyl)acetyl]-6-(6-methyl-pyridin-2-yl-1H-[1,2,3]-triazol-4-yl)-3,4-dihydro-2H-benzo[1,4]-oxazine (Example 17) and pharmaceutically acceptable derivatives thereof.

11. A pharmaceutical composition comprising a compound according to claim 1, or a pharmaceutically acceptable derivative thereof, and a pharmaceutically acceptable carrier or diluent.

12-13. (canceled)

14. A method for the treatment or prophylaxis of kidney fibrosis comprising, administering to a subject in need thereof, a compound according to claim 1, or a pharmaceutically acceptable derivative thereof.
